(19) United States
(12) Patent Application Publication
(10) Pub. No.: US 2025/0221415 A1
(43) Pub. Date: Jul. 10, 2025

(54) Materials and Methods for Treating Iron Deficiencies and Iron Chlorosis in Plants

(71) Applicant: Locus Solutions IPCo, LLC, Solon, OH (US)

(72) Inventor: Maja MILOVANOVIC, North Royalton, OH (US)

(21) Appl. No.: 18/843,771

(22) PCT Filed: Jul. 7, 2023
(86) PCT No.: PCT/US2023/069776

§ 371 Date: Sep. 4, 2024

Related U.S. Application Data
(60) Provisional application No. 63/359,248, filed on Jul. 8, 2022.

(51) Int. Cl. A01N 63/22; A01P 21/00; C12N 1/20; C12R 1/07

(52) U.S. Cl. CPC A01N 63/22; A01P 21/00; C12N 1/205; C12R 2001/07

(57) ABSTRACT

The subject invention provides compositions and methods for treating and/or preventing iron deficiencies and iron chlorosis in plants. Compositions comprising an iron-capturing ingredient are contacted with plant and/or its surrounding environment in order to increase the amount of iron that is bioavailable to the plant.

### CROSS-REFERENCE TO RELATED APPLICATION

This application claims priority to U.S. Provisional Application No. 63/359,248, filed Jul. 8, 2022, which is incorporated herein by reference in its entirety.

### BACKGROUND OF THE INVENTION

In plants, iron is required for photosynthesis and chlorophyll synthesis. The availability of iron in soils can dictate the distribution of plant species in natural ecosystems and limit the yield and nutritional quality of crops. Insufficient iron uptake causes reduced growth, interveinal chlorosis, and reduced overall fitness. Sufficient iron levels in food crops are important to combat iron deficiency-induced anemia in people, which is one of the largest nutritional disorders worldwide.

When plants are deficient in iron, the result can be a condition known as iron chlorosis, which exhibits as a yellowing of plant leaves. The primary symptom of iron deficiency is interveinal chlorosis, or the development of a yellow leaf with dark green veins. In severe cases, the entire leaf can turn yellow or white as the plant cells die. It is common for only a branch or half of a tree to be chlorotic while the other part of the tree remains normal.

Yellow leaves indicate a lack of chlorophyll in the plant. A reduction in chlorophyll during the growing season can negatively impact the plant's growth and vigor. If a food crop is chlorotic, it can produce smaller fruits with a bitter flavor.

Iron chemistry in soil is complex, and various reactions in the soil can lead to low iron bioavailability and increased cases of iron chlorosis. Iron chlorosis often occurs in soils that are alkaline (pH greater than 7.0) and that contain lime. Even though there can be adequate levels of iron these soils, it has low solubility, particularly in high soil pH soils.

Further aggravating factors that can lead to iron deficiency and chlorosis include reduced soil temperatures and conditions that restrict air movement into the soil, such as, for example, plastic sheet mulching, compaction, and water-saturated conditions. Chlorosis can be more severe in places where topsoil has been removed, exposing lime enriched subsoil, such as, for example, areas with eroded soils or soils subjected to land leveling.

Some plants have evolved adaptations to living in high pH soils with deficient and/or low-solubility iron, for example, by mobilizing iron by secreting iron chelating substances, increasing iron storage in cells, and optimizing photosynthesis to protect from absorbing too much light energy.

Nonetheless, not all plants have these adaptations, meaning growers must utilize other measures to address iron deficiency in crops. Such measures can include, for example, adding manure and fertilizer to soil, as well as spraying fertilizer directly onto plant leaves. While these can be short-term fixes, when chemical fertilizers are overused or improperly applied, they can run off into surface water, leach into groundwater, and evaporate into the air. As sources of air and water pollution, these substances are increasingly scrutinized, making their responsible use an ecological and commercial imperative. Even when properly used, the over-dependence and long-term use of certain chemical fertilizers can deleteriously alter soil ecosystems.

Mounting regulatory mandates governing the availability and use of chemicals and consumer demands for residue free, sustainably-grown food which production has minimal impact on the environment in which that food and fiber is grown are impacting the industry and causing an evolution of thought regarding how to address the myriad of challenges. Accordingly, effective and environmentally-friendly methods and compositions are needed to for addressing nutrient deficiencies in plants, including treating and/or preventing iron deficiencies and iron chlorosis in plants.

### BRIEF SUMMARY OF THE INVENTION

The subject invention pertains to methods and compositions for treating nutrient deficiencies in plants. More specifically, the subject invention provides compositions and methods for increasing the amount of bioavailable iron for plants to absorb and utilize. Advantageously, the compositions and methods of the subject invention are environmentally-friendly, non-toxic and cost-effective agricultural solutions to improve plant health, growth and/or yields.

In preferred embodiments, the subject invention provides methods for treating and/or preventing a nutrient deficiency in a plant, wherein a composition comprising an iron-capturing ingredient is administered to the plant and/or into an environment in which the plant is growing. In a specific embodiment, the plant has been determined to be afflicted with iron deficiency and/or iron chlorosis.

The subject invention further provides a composition comprising one or more ingredients that capture iron. In certain embodiments, the iron-capturing ingredient is a beneficial microorganism, a growth by-product of an iron-capturing microorganism, or some other compound capable of binding iron and improving its solubility and/or bioavailability for plants. In certain embodiments, more than one iron-capturing ingredient is included in the composition.

In some embodiments, the method is carried out at pH less than 6.8, preferably, less than 5.0, more preferably less than 4.8. Thus, in some embodiments, the method further comprising administering the composition in combination with an acidic pH adjuster.

In preferred embodiments, the beneficial microorganisms of the subject invention are non-pathogenic fungi, yeasts and/or bacteria capable of sequestering iron, either naturally or through genetic modification.

In one embodiment, the beneficial microorganism is a strain of Bacillus subtilis. In a specific embodiment, the strain is B. subtilis B4 (NRRL B-68031). The B4 strain is preferably administered in spore form but grows in biofilm form when exposed to acidic environments.

Surprisingly, B4 was found to produce one or more compounds capable of sequestering, chelating or otherwise capturing iron. In certain embodiments, the compounds are pulcherrimin and/or pulcherriminic acid. Advantageously, the microbes and/or the exopolysaccharide (EPS) of the microbes when grown in biofilm form effectively assimilate iron using an iron-capturer such as pulcherrimin and/or pulcherriminic acid upon exposure to an acidic pH (e.g., less than 6.8, preferably less than 5.0). In some embodiments, the iron is then made available to the plant as a nutrient. This can be facilitated by shifting the pH towards a more neutral pH (e.g., 6.8-8.0).

B4 is also particularly advantageous over other traditional probiotic microorganisms due to its ability to produce digestive enzymes, including, for example, cellulases and amylases.

In certain embodiments, the composition can comprise other non-pathogenic microorganisms that are capable of producing compounds that can sequester, chelate or otherwise capture iron. The microorganism(s) can be in biofilm form, spore form, planktonic form, or any other form.

In certain embodiments, the microorganisms are also capable of producing one or more of the following: surface active agents, such as lipopeptides and/or glycolipids; bioactive compounds with antimicrobial and immune-modulating effects; polyketides; acids; peptides; anti-inflammatory compounds; enzymes, such as amylases, cellulases, proteases and/or lipases; and sources of amino acids, vitamins, and other nutrients.

In certain embodiments, the iron-capturing ingredient of the subject composition is a crude form or purified siderophore or phytosiderophore, or other molecule with high iron affinity, for example, pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexanediamide (DPH), 2,3-dihydroxybenzoic acid, azotochelin, transferrin, enterobactin, pyoverdine, protochelin, pyochelin, bacillibactin, vibriobactin, vibrioferrin azotobactin, aminochelin, yersiniabactin, agrobactin, staphyloferrin, ferrichrome, defarasirox, deferiprone, desferrioxamine, fusarinine, chrysobactin, achromobactin, ornibactin, rhodotorulic acid, lysine, glutamic acid, gluconic acid, iron oxyhydroxide minerals, ferrihydrite, magnetite, hematite, gcothite, sideritchydroxamate, catecholates, salicylates, carboxylates, mugineic acid, ferulic acid, caffeic acid, and/or nicotianamine.

In certain embodiments, the composition comprises an organic or inorganic acid. Preferably, the acid is present in an amount suitable for adjusting the pH of the composition or the environment to which it is applied to 6.8 or lower, preferably, 5.0 or lower, more preferably, 4.8 or lower.

The subject methods can be used to inoculate a plant's rhizosphere with one or more beneficial microorganisms. For example, the microbes of the subject composition can colonize the rhizosphere and provide multiple benefits to a plant whose roots are growing therein, including protection, nourishment, and increased iron uptake.

The subject methods can also be used to improve the health of soil by, for example, improving water and nutrient dispersion, and/or reducing compaction.

Furthermore, in certain embodiments, the subject methods can be used to enhance health, growth and/or yields in plants having compromised health due to lack of iron. Thus, in certain embodiments, the subject methods can also be used for improving the health, or immune response, of plants.

Advantageously, the present invention can be used without releasing large quantities of inorganic compounds into the environment. Additionally, the compositions and methods utilize components that are biodegradable and toxicologically safe. Thus, the present invention can be used as a “green” soil and plant treatment.

### BRIEF DESCRIPTION OF THE FIGURES

FIGS. 1A-1C show growth and exopolysaccharide (EPS) formation of B4 after 24 hours (A) and 48 hours (B) at acidic pH (top row, pH 4.8) and neutral pH (bottom row, pH 6.8).

FIG. 2 shows purified B4 EPS with a pink hue.

FIG. 3 shows result of an amylase test for B4. Agar streaked with B4 produced an orange color around the bacterial growth, indicating the breakdown of starch.

FIG. 4 show results of a cellulase test for B4. Agar streaked with B4 produced a yellow zone of clearing around the bacterial growth, indicating the breakdown of cellulose.

FIGS. 5A-5C show B4 siderophore production and activity after 6 hours in aerobic (top plates) and anaerobic (bottom plates) environments and on different growth media. (A) shows B4 culture grown in MRS-sucrose (left side) and M23-6 (right side) media. (B) shows B4 culture grown in minimal media with Tween (left side) and minimal media without Tween (right side). (C) shows dried B4 spores grown in minimal medium.

FIGS. 6A-6C show B4 siderophore production and activity after 24 hours in aerobic (top plates) and anaerobic (bottom plates) environments and on different growth media. (A) shows B4 culture grown in M23-6 (left side) and MRS-sucrose (right side) media. (B) shows B4 culture grown in minimal media with Tween (left side) and minimal media without Tween (right side). (C) shows dried B4 spores grown in minimal medium.

FIG. 7 shows results of an iron assay for B4 (BSSL) cultures grown in different media.

### DETAILED DESCRIPTION OF THE INVENTION

The subject invention provides compositions and methods for treating and/or preventing iron deficiencies in plants. More specifically, the subject invention provides compositions that can be applied as an iron chelate to the plant leaves and/or to the soil in which the plants grow in order to increase the amount of bioavailable iron for the plants to utilize. Advantageously, the microbe-based products and methods of the subject invention are environmentally-friendly, non-toxic and cost-effective.

### Selected Definitions

As used herein, a “biofilm” is a complex aggregate of microorganisms, such as bacteria, wherein the cells adhere to each other and/or to a surface. In certain embodiments, adherence is achieved via an exopolysaccharide substance produced by the bacteria. The cells in biofilms are physiologically distinct from planktonic cells of the same organism, which are single cells that can float or swim in liquid medium.

As used herein “preventing” or “prevention” of a disease, condition or disorder means delaying, inhibiting, suppressing, forestalling, and/or minimizing the onset or progression of a particular sign or symptom thereof. Prevention can include, but does not require, indefinite, absolute or complete prevention, meaning the sign or symptom may still develop at a later time. Prevention can include reducing the severity of the onset of such a disease, condition or disorder, and/or inhibiting the progression of the condition or disorder to a more severe condition or disorder.

As used herein, “treating” or “treatment” of a disease, condition or disorder means the eradicating, improving, reducing, ameliorating or reversing of at least one sign or symptom of the disease, condition or disorder (e.g., an infection). Treatment can include, but does not require, a complete cure of the disease, condition or disorder, meaning treatment can also include partial eradication, improvement, reduction, amelioration or reversal.

As used herein, an “isolated” or “purified” nucleic acid molecule, polynucleotide, polypeptide, protein, organic compound such as a small molecule (e.g., those described below), or other compound is substantially free of other compounds, such as cellular material, with which it is associated in nature. For example, a purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. A purified or isolated microbial strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, “ionophores” are carboxylic polyether non-therapeutic antibiotics that disrupt the ion concentration gradient (Ca2+, K+, H+, Na+) across microorganisms, which causes them to enter a futile ion cycle. The disruption of the ion concentration prevents the microorganism from maintaining normal metabolism and causes the microorganism to expend extra energy. Ionophores function by selecting against or affecting the metabolism of gram-positive bacteria, such as methanogens, and protozoa.

As used herein, “siderophores” are compounds produced by different organisms for the purpose of scavenging iron from the surrounding environment. Siderophores are typically small, low molecular weight compounds with high affinity for ferric iron (Fe³⁺), forming strong ferric chelate complexes that can, in some instances be taken up by the organisms. As used herein, “phytosiderophores” are siderophores produced by plants.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites can include, but are not limited to, enzymes, toxins, acids, solvents, alcohols, proteins, carbohydrates, vitamins, minerals, microelements, amino acids, polymers, polyketides, and surfactants.

As used herein, a “methanogen” is a microorganism that produces methane gas as a by-product of metabolism. Methanogens are archaea that can be found in the digestive systems and metabolic waste of ruminant animals and non-ruminant animals (e.g., pigs, poultry and horses). Examples of methanogens include, but are not limited to, Methanobacterium spp. (e.g., M. formicicum), Methanobrevibacter spp. (e.g., M. ruminantium), Methanococcus spp. (e.g., M. paripaludis), Methanoculleus spp. (e.g., M. bourgensis), Methanoforens spp. (e.g., M. stordalenmirensis), Methanofollis liminatans, Methanogenium wolfei, Methanomicrobium spp. (e.g., M. mobile), Methanopyrus kandleri, Methanoregula boonei, Methanosaeta spp. (e.g., M. concilii, M. thermophile), Methanosarcina spp. (e.g., M. barkeri, M. mazeii), Methanosphaera stadtmanae, Methanospirillium hungatei, Methanothermobacter spp., and/or Methanothrix sochngenii.

As used herein, “surfactant” refers to a surface-active compound that lowers the surface tension (or interfacial tension) between two liquids, between a liquid and a gas, and/or between a liquid and a solid. Surfactants act as, e.g., detergents, wetting agents, emulsifiers, foaming agents, and dispersants. A “biosurfactant” is a surface active molecule produced by a living organism and/or using naturally-derived substrates.

As used herein, “agriculture” means the cultivation and breeding of plants, algae and/or fungi for food, fiber, biofuel, medicines, cosmetics, supplements, ornamental purposes and other uses. According to the subject invention, agriculture can also include horticulture, landscaping, gardening, plant conservation, orcharding and arboriculture. Further included in agriculture are the care, monitoring and maintenance of soil.

As used herein, the term “plant” includes, but is not limited to, any species of woody, ornamental or decorative, crop or cereal, fruit plant or vegetable plant, flower or tree, macroalga or microalga, phytoplankton and photosynthetic algae (e.g., green algae Chlamydomonas reinhardtii). “Plant” also includes a unicellular plant (e.g., microalga) and a plurality of plant cells that are largely differentiated into a colony (e.g., volvox) or a structure that is present at any stage of a plant's development. Such structures include, but are not limited to, a fruit, a seed, a shoot, a stem, a leaf, a root, a flower petal, etc. Plants can be standing alone, for example, in a garden, or can be one of many plants, for example, as part of an orchard, crop or pasture.

As used herein, “crop plants” refer to any species of plant or alga, grown for profit and/or for sustenance for humans, animals or aquatic organisms, or used by humans (e.g., textile, cosmetics, and/or drug production), or viewed by humans for pleasure (e.g., flowers or shrubs in landscaping or gardens) or any plant or alga, or a part thereof, used in industry, commerce or education. Crop plants can be plants that can be obtained by traditional breeding and optimization methods or by biotechnological and recombinant methods, or combinations of these methods, including the transgenic plants and the plant varieties.

All plants and plant parts can benefit from the subject invention. In this context, plants are understood as meaning all plants and plant populations such as desired and undesired wild plants or crop plants (including naturally occurring crop plants).

Plant tissue and/or plant parts are understood as meaning all aerial and subterranean parts and organs of the plants such as shoots, leaves, flowers, roots, needles, stalks, stems, fruits, seeds, tubers and rhizomes. The plant parts also include crop material and vegetative and generative propagation material, for example cuttings, tubers, rhizomes, slips and seeds. As used herein, “enhancing” means improving or increasing. For example, enhanced plant health means improving the plant's ability grow and thrive (which includes increased seed germination, seedling emergence, and/or vigor); improved ability to withstand transplant shock; improved ability to ward off pests and/or diseases; improved ability to compete with weeds; and improved ability to survive environmental stressors, such as droughts and/or overwatering.

Enhancing plant growth and/or enhanced plant biomass means, for example, increasing the size and/or mass of a plant both above and below the ground (e.g., increased canopy/foliar volume, bud size, height, trunk caliper, branch length, shoot length, stalk length, protein content, root size/density and/or overall growth index), and/or improving the ability of the plant to reach a desired size and/or mass.

Enhancing yields mean, for example, improving the end products produced by the plants in a crop, for example, by increasing the amount, number and/or size of fruits, leaves, roots, flowers, buds, stalks, seeds, fibers, extracts and/or tubers per plant, and/or improving the quality thereof.

As used herein, a “soil amendment” or a “soil conditioner” is any compound, material, or combination of compounds or materials that are added into soil to enhance the physical properties of the soil. Soil amendments can include organic and inorganic matter, and can further include, for example, fertilizers, pesticides and/or herbicides. Nutrient-rich, well-draining soil is essential for the growth and health of plants, and thus, soil amendments can be used for enhancing the growth and health of plants by altering the nutrient and moisture content of soil. Soil amendments can also be used for improving many different qualities of soil, including but not limited to, soil structure (e.g., preventing compaction); improving the nutrient concentration and storage capabilities; improving water retention in dry soils; and improving drainage in waterlogged soils.

As used herein, an “abiotic stressor” refers to a non-living condition that has a negative impact on a living organism in a specific environment. The abiotic stressor must influence the environment beyond its normal range of variation to adversely affect the population performance or individual physiology of the organism. Examples of abiotic stressors include, but are not limited to, drought, extreme temperatures, flood, high winds, natural disasters, soil pH changes, high radiation, compaction of soil, pollution, and others. A “biotic stressor” is one caused by a living condition, for example, an animal, plant, or microbial pest.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduction” means a negative alteration and “increase” means a positive alteration, wherein the positive or negative alteration is at least 0.25%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

The transitional term “comprising,” which is synonymous with “including,” or “containing,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Compositions

The subject invention provides a composition for use according to the subject methods, wherein the composition comprises one or more ingredients that capture iron. In certain embodiments, the iron-capturing ingredient is a beneficial microorganism, a growth by-product of a microorganism, or some other compound known to bind iron. In certain embodiments, more than one iron-capturing ingredient is included in the composition.

The total iron-capturing ingredient(s) in the composition preferably comprise from 0.0001% to 100% of the composition by weight or by volume, or from 0.001 to 95%, from 0.01 to 90%, from 0.1% to 85%, from 0.5 to 80%, from 0.75 to 75%, from 1.0 to 70%, from 1.25 to 65%, from 1.5 to 60%, from 1.75 to 55%, from 2.0 to 50%, or from 5.0 to 25% by weight or by volume.

In certain embodiments, the composition is a “microbe-based composition,” meaning a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of microbial propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. The cells may be totally absent, or present at, for example, a concentration of at least 1×10⁴, 1×10⁵, 1×10⁶, 1×10⁷, 1×10⁸, 1×10⁹, 1×10¹⁰, 1×10¹¹, 1×10¹², 1×10¹³ or more CFU per milliliter or CFU/g of the composition.

Advantageously, in preferred embodiments, the subject compositions can sequester iron in soil and make the iron available to a plant in need thereof. Thus, in some embodiments, the composition can be used for treating an iron deficiency and/or iron chlorosis in plants.

In preferred embodiments, the beneficial microorganisms of the subject compositions are non-pathogenic fungi, yeasts and/or bacteria capable of sequestering iron, either naturally or through genetic modification. The beneficial microorganisms may be in an active, inactive and/or dormant form. In preferred embodiments, the microorganism is one that is characterized as “generally regarded as safe,” or GRAS, by the appropriate regulatory agency.

In certain embodiments, the microorganisms are also capable of producing one or more of the following: surface active agents, such as lipopeptides and/or glycolipids; bioactive compounds with antimicrobial and immune-modulating effects; polyketides; acids; peptides; anti-inflammatory compounds; enzymes, such as amylases, cellulases, proteases and/or lipases; and sources of amino acids, vitamins, and other nutrients.

The microorganisms of the subject invention may be natural, or genetically modified microorganisms. For example, the microorganisms may be transformed with specific genes to exhibit specific characteristics. The microorganisms may also be mutants of a desired strain. As used herein, “mutant” means a strain, genetic variant, or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In some embodiments, the beneficial microorganisms are selected based on a natural or acquired resistance to certain antibiotics administered to an environment comprising an iron-capturing pathogen to, for example, control pathogenic and/or deleterious microbes in a living subject or elsewhere in an environment.

In one specific embodiment, the composition comprises about 1×10⁶ to about 1×10¹³, about 1×10⁷ to about 1×10¹², about 1×10⁸ to about 1×10¹¹, or about 1×10⁹ to about 1×10¹⁰ CFU/g of each species of microorganism present in the composition.

In one embodiment, the composition comprises about 0.001 to 100% microorganisms total by volume, about 1 to 90%, or about 10 to 75%.

In certain embodiments, the composition comprises a growth by-product of a microorganism but no living microorganism. For example, in certain embodiments, a pathogenic microorganism is utilized only in the production of growth by-products for producing a composition according to the subject invention as opposed to direct administration to an environment.

The microorganisms can include yeasts, bacteria and/or fungi, including, for example, Acaulospora, Acidithiobacillus spp. (e.g., A. ferooxidans, A. albertensis, A. caldus, A. cuprithermicus, A. ferrianus, A. ferridurans, A. ferriphilus, A. ferrivorans, A. ferrooxidans, A. sulfuriphilus, and A. thiooxidans), Acremonium chrysogenum, Agrobacterium (e.g., A. radiobacter), Aspergillus, Aureobasidium (e.g., A. pullulans), Azospirillum (e.g., A. brasiliensis), Azotobacter (A. vinelandii, A. chroococcum), Bacillus (e.g., B. amyloliquefaciens, B. coagulans, B. firmus, B. laterosporus, B. licheniformis, B. megaterium, B. mucilaginosus, B. subtilis), Blakeslea, Candida (e.g., C. albicans, C. apicola, C. batistae, C. bombicola, C. floricola, C. kuoi, C. riodocensis, C. nodaensis, C. stellate), Cryptococcus, Debaryomyces (e.g., D. hansenii), Dipodascopsism, Entomophthora, Escherichia coli, Frateuria (e.g., F. aurantia), Hanseniaspora (e.g., H. uvarum), Hansenula, Issatchenkia, Kluyveromyces (e.g., K. phaffii), Lentinula spp. (e.g., L. edodes), Legionella pneumophila, Lipomyces, Magnetospirillum magneticum, Magnetococcus marinus, methanogens, Metschnikowia sp. (M. pulcherrimia), Meyerozyma (e.g., M. guilliermondii, M. caribbica), Monascus purpureus, Mortierella, Mucor (e.g., M. piriformis), Neisseria meningitidis, Pantoea (e.g., P. agglomerans, P. allii), Penicillium, Phythium, Phycomyces, Pichia (e.g., P. anomala. P. guilliermondii, P. occidentalis, P. kudriavzevii), Pleurotus (e.g., P. ostreatus P. ostreatus, P. sajorcaju, P. cystidiosus, P. cornucopiae, P. pulmonarius, P. tuberregium, P. citrinopileatus and P. flabellatus), Pseudomonas (e.g., P. chlororaphis, P. aeruginosa, P. koreensis), Pseudozyma (e.g., P. aphidis, P. antarctica), Rhizobium radiobacter, Rhizopus, Rhodospirillum (e.g., R. rubrum), Rhodotorula (e.g., R. bogoriensis), Saccharomyces (e.g., S. cerevisiae, S. boulardii, S. torula), Sphingomonas (e.g., S. paucimobilis), Starmerella (e.g., S. bombicola), Streptomyces, Torulopsis, Thraustochytrium, Trichoderma (e.g., T. reesei, T. harzianum, T. viridae), Ustilago (e.g., U. maydis), Vibrio cholerae, Wickerhamiella (e.g., W. domericqiue), Wickerhamomyces (e.g., W. anomalus), Williopsis (e.g., W. mrakii), Zygosaccharomyces (e.g., Z. bailii), and others (including those listed as pathogens elsewhere in this disclosure).

If present, fungi can be in the form of live or inactive cells, mycelia, spores and/or fruiting bodies. The fruiting bodies, if present, can be, for example, chopped and/or blended into granules and/or a powder form.

If present, yeasts can be in the form of live or inactive cells or spores, as well as in the form of dried and/or dormant cells (e.g., a yeast hydrolysate).

If present, bacteria can be in the form of vegetative or planktonic cells, biofilms, spores, and/or a dried cell or spore mass.

In some embodiments, dried microbes, e.g., spores, can be mixed with fillers known in the art, such as e.g., microcrystalline cellulose (MCC).

In one embodiment, the composition comprises one or more Bacillus spp. bacteria and/or growth by-products thereof. In certain embodiments, the Bacillus spp. are B. amyloliquefaciens, B. subtilis, B. coagulans and/or B. licheniformis.

In one embodiment, the composition comprises B. amyloliquefaciens NRRL B-67928 “B. amy” and/or a growth by-product thereof. A culture of the B. amyloliquefaciens “B. amy” microbe has been deposited with the Agricultural Research Service Northern Regional Research Laboratory (NRRL) Culture Collection, 1815 N. University St., Peoria, IL, USA. The deposit has been assigned accession number NRRL B-67928 by the depository and was deposited on Feb. 26, 2020.

In one embodiment, the composition comprises a strain of Bacillus subtilis and/or a growth by-product thereof. In a specific embodiment, the strain is B. subtilis B4 (NRRL B-68031). A culture of the B4 microbe has been deposited with the Agricultural Research Service Northern Regional Research Laboratory (NRRL) Culture Collection, 1815 N. University St., Peoria, IL, USA. The deposit has been assigned accession number NRRI B-68031 by the depository and was deposited on May 6, 2021.

B4 is a Gram-positive spore-forming strain of B. subtilis that is capable of anaerobic growth (obligate anaerobe). The B4 strain is preferably administered in spore form but germinates in acidic environments, wherein it can grow in biofilm form. Surprisingly, B4 was found to produce one or more compounds capable of sequestering, chelating or otherwise capturing iron when grown in biofilm form. In certain embodiments, the compounds are pulcherrimin and/or pulcherriminic acid.

Advantageously, the microbes and/or the exopolysaccharide (EPS) of the biofilm effectively assimilate iron using an iron-capturer such as, e.g., pulcherrimin and/or pulcherriminic acid, while traveling through low pH (e.g., less than 6.8, less than 5.0, or less than 4.8). The iron can then be made more bioavailable to a plant by the microbe once, for example, the pH raises to a slightly more neutral pH (e.g., 6.8-8.0).

B4 is also particularly advantageous over other agricultural microorganisms due to its ability to produce increased amounts of the lipopeptide surfactin (e.g., greater than wild type B. subtilis), as well as digestive enzymes, including, for example, cellulases and amylases. These enzymes help digest nutrient matter into smaller units, such as volatile fatty acids (e.g., propionate, acetate, butyrate), glucose and amino acids.

The proprietary cultures described herein have been deposited under conditions that assure that access to the cultures will be available during the pendency of this patent application to one determined by the Commissioner of Patents and Trademarks to be entitled thereto under 37 CFR § 1.14 and 35 U.S.C § 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, each of the subject culture deposits will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

In certain embodiments, the composition can comprise other microorganisms that are capable, either naturally or by genetic modification, of producing pulcherrimin and/or pulcherriminic acid, or other compounds capable of sequestering, chelating or otherwise capturing iron. In specific embodiments, the microbes are capable of growing as a biofilm.

In certain embodiments, the microorganism is a naturally-occurring or genetically-modified microorganism capable of regulating genes involved in iron capture and transport, e.g., HFE, GDF15, TWSG1, ERFE, Matriptase 2, TF, TFR1, TFR2, HAMP and HJV.

In certain embodiments, the composition can comprise a crude form or purified siderophore or phytosiderophore, or other molecule with high iron affinity, for example, pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexanediamide (DPH), 2,3-dihydroxybenzoic acid, azotochelin, transferrin, enterobactin, pyoverdine, protochelin, pyochelin, bacillibactin, vibriobactin, vibrioferrin azotobactin, aminochelin yersiniabactin, agrobactin, staphyloferrin, ferrichrome, defarasirox, deferiprone, desferrioxamine, fusarinine, chrysobactin, achromobactin, ornibactin, rhodotorulic acid, lysine, glutamic acid, gluconic acid, iron oxyhydroxide minerals, ferrihydrite, magnetite, hematite, gcothite, sideritehydroxamate, catecholates, salicylates, carboxylates, mugineic acid, ferulic acid, caffeic acid, and/or nicotianamine.

The composition can also comprise other microbial growth by-products. The microbial growth by-product can be produced by the microorganisms of the composition, and/or they can be produced separately, e.g., by a microorganism listed herein, and added to the composition.

In certain embodiments, the composition can comprise substrate leftover from cultivation, and/or purified or unpurified growth by-products, such as biosurfactants, killer toxins, enzymes, polyketides, and/or other metabolites. The microbes can be live or inactive, although, in preferred embodiments, if the microbe is considered a pathogen, the microbe is inactivated and/or removed from the composition.

In one embodiment, the growth by-product has been purified from the cultivation medium in which it was produced. Alternatively, in one embodiment, the growth by-product is utilized in crude form. The crude form can comprise, for example, a liquid supernatant resulting from cultivation of a microbe that produces the growth by-product of interest, including residual cells and/or nutrients.

The growth by-products can include metabolites or other biochemicals produced as a result of cell growth, including, for example, amino acids, peptides, polyketides, antibiotics, proteins, enzymes, biosurfactants, solvents, vitamins, and/or other metabolites.

### Additional Components

Further components can be added to the composition, for example, carriers, other microbe-based compositions, biosurfactants, enzymes, catalysts, solvents, buffers, emulsifying agents, lubricants, solubility controlling agents, preservatives, stabilizers, ultra-violet light resistant agents, viscosity modifiers, preservatives, tracking agents, biocides, surfactants, pH adjusting agents, essential oils, botanical extracts, cross-linking agents, chelators, fatty acids, alcohols, reducing agents, syndetics, dyes, colorants, fragrances, antimicrobial compounds, antibiotics, foaming agents, foam reducers, polymers, thickeners, chelators, and other ingredients specific for an intended use.

In certain specific embodiments, the composition comprises a chelating agent including, but are not limited to, ethylenediaminetetraacetic acid (EDTA), nitrilotriacetic acid (NTA), a phosphonate, succimer (DMSA), diethylenetriaminepentaacetate (DTPA), N-acetylcysteine, n-hydroxyethylethylenediaminetriacetic acid (HEDTA), organic acids with more than one coordination group (e.g., rubcanic acid), STPP (sodiumtripolyphosphate, Na5P3O10), trisodium phosphate (TSP), water, carbohydrates, organic acids with more than one coordination group (e.g., citric acid), lipids, steroids, amino acids or related compounds (e.g., glutathione), peptides, phosphates, nucleotides, tetrapyrrols, ferrioxamines, ionophores, orphenolics, sodium citrate, sodium gluconate, ethylenediamine disuccinic acid (EDDS), iminodisuccinic acid (IDS), L-glutamic acid diacetic Acid (GLDA), GLDA-Na4, methyl glycindiacetic acid (MGDA), polyaspartic acid (PASA), hemoglobin, chlorophyll, lipophilic β-diketone, and (14,16)-hentriacontanedione, ethylenediamine-N,N′-diglutaric acid (EDDG), ethylenediamine-N,N′-dimalonic acid (EDDM), 3-hydroxy-2,2-iminodisuccinic acid (HIDS), 2-hydroxyethyliminodiacetic acid (HEIDA), pyridine-2,6-dicarboxylic acid (PDA), trimethyl glycine (TMG), Tiron, or any combination thereof.

In certain embodiments, the composition comprises a germination enhancer for enhancing germination of spore-form microorganisms used in the microbe-based composition. In specific embodiments, the germination enhancers are amino acids, such as, for example, L-alanine and/or L-leucine. In one embodiment, the germination enhancer is manganese.

In certain embodiments, the composition comprises an organic acid selected from, for example, acetic acid, citric acid, formic acid, lactic acid, malic acid, oxalic acid, tartaric acid, uric acid, propionic acid, butyric acid, sorbic acid, fumaric acid, benzoic acid, hydrofluoric acid, caproic acid, salicylic acid, gluconic acid, pyruvic acid, adipic acid, trichloroacetic acid, glycolic acid, cinnamic acid, carboxylic acids, succinic acid, carbonic acid, glutaric acid, decanoic acid, and ascorbic acid. In some embodiments, the composition comprises an inorganic acid selected from, for example, hydrochloric acid, sulfuric acid, nitric acid, phosphoric acid, boric acid, perchloric acid, hydrofluoric acid, hydrobromic acid, and sulfonic acid. Preferably, the acid is present in an amount suitable for adjusting the pH of the composition or the environment to which it is applied to 6.8 or lower, preferably, 5.0 or lower, more preferably, 4.8 or lower.

In one embodiment, the composition comprises one or more fatty acids. The fatty acids can be produced by the microorganisms of the composition, and/or produced separately and included as an additional component. In certain preferred embodiments, the fatty acid is a saturated long-chain fatty acid, having a carbon backbone of 14-20 carbons, such as, for example, myristic acid, palmitic acid, or stearic acid. In some embodiments, a combination of two or more saturated long-chain fatty acids is included in the composition. In some embodiments, a saturated long-chain fatty acid can inhibit methanogenesis and/or increase cell membrane permeability of methanogens.

In certain embodiments, the composition comprises one or more enzymes that help digest food sources into smaller units, such as volatile fatty acids (e.g., propionate, acetate, butyrate), glucose and amino acids. These enzymes can be produced by the microorganisms of the composition, and/or produced separately and included as an additional component. Non-limiting examples of digestive enzymes include amylases, maltases, lactases, lipases, proteases, sucrases and cellulases.

In some embodiments, the composition can comprise additional components known to reduce methane production from methanogens, such as, for example, nitrates (e.g., calcium nitrate, ammonium nitrate, sodium nitrate, potassium nitrate, and magnesium nitrate); seaweed (e.g., Asparagopsis taxiformis and/or Asparagopsis armata); kelp; nitrooxypropanols (e.g., 3-nitrooxypropanol and/or ethyl-3-nitrooxypropanol); anthraquinones; ionophores (e.g., monensin and/or lasalocid); polyphenols (e.g., saponins, tannins); Yucca schidigera extract (steroidal saponin-producing plant species); Quillaja saponaria extract (triterpenoid saponin-producing plant species); organosulfurs (e.g., garlic extract); flavonoids (e.g., quercetin, rutin, kaempferol, naringin, and anthocyanidins; bioflavonoids from green citrus fruits, rose hips and black currants); carboxylic acid; and/or terpenes (e.g., d-limonene, pinene and citrus extracts).

In one embodiment, the composition can comprise one or more biosurfactants. Biosurfactants are a structurally diverse group of surface-active substances produced by microorganisms, which are biodegradable and can be efficiently produced using selected organisms on renewable substrates. All biosurfactants are amphiphiles. They consist of two parts: a polar (hydrophilic) moiety and non-polar (hydrophobic) group. The common lipophilic moiety of a biosurfactant molecule is the hydrocarbon chain of a fatty acid, whereas the hydrophilic part is formed by ester or alcohol groups of neutral lipids, by a carboxylate group of fatty acids or amino acids (or peptides), an organic acid in the case of flavolipids, or, in the case of glycolipids, by a carbohydrate.

Due to their amphiphilic structure, biosurfactants increase the surface area of hydrophobic water-insoluble substances, increase the water bioavailability of such substances, and change the properties of bacterial cell surfaces. Biosurfactants accumulate at interfaces, thus reducing interfacial tension and leading to the formation of aggregated micellar structures in solution. Safe, effective microbial biosurfactants reduce the surface and interfacial tensions between the molecules of liquids, solids, and gases. The ability of biosurfactants to form pores and destabilize biological membranes permits their use as antibacterial, antifungal, and hemolytic agents.

Advantageously, in certain embodiments, biosurfactants can help disrupt and/or penetrate biofilms for increased effectiveness of antibacterial compounds. Furthermore, in certain embodiments, biosurfactants can facilitate the increase in transport and/or bioavailability of nutrients through and into cells, including into plant roots.

Biosurfactants according to the subject invention can include, for example, glycolipids, lipopeptides, flavolipids, phospholipids, fatty acid esters, and high molecular weight polymers such as lipoproteins, lipopolysaccharide-protein complexes, and polysaccharide-protein-fatty acid complexes.

In one embodiment, the biosurfactant is a glycolipid. Glycolipids can include, for example, sophorolipids, rhamnolipids, cellobiose lipids, mannosylerythritol lipids and trehalose lipids. In one embodiment, the biosurfactant is a lipopeptide. Lipopeptides can include, for example, surfactin, iturin, arthrofactin, viscosin, fengycin, and lichenysin. In certain embodiments, a mixture of biosurfactants is used.

In one embodiment, the biosurfactant has been purified from the fermentation medium in which it was produced. Alternatively, in one embodiment, the biosurfactant is utilized in crude form comprising fermentation broth resulting from cultivation of a biosurfactant-producing microbe. This crude form biosurfactant solution can comprise from about 0.001% to 99%, from about 25% to about 75%, from about 30% to about 70%, from about 35% to about 65%, from about 40% to about 60%, from about 45% to about 55%, or about 50% pure biosurfactant, along with residual cells and/or nutrients.

In one embodiment, the composition comprises a saponin at 1 to 10 ml/L, or 2 to 6 ml/L. of ruminal fluid. Saponins are natural surfactants that are found in many plants and that exhibit similar characteristics to microbial biosurfactants, for example, self-association and interaction with biological membranes. There are three basic categories of saponins, including triterpenoid saponins, steroidal saponins, and steroidal glycoalkaloids.

Some well-known triterpenoid saponin-accumulating plant families include the Leguminosae, Amaranthaceae, Apiaceae, Caryophyllaceae, Aquifoliaceae, Araliaceae, Cucurbitaceae, Berberidaceae, Chenopodiaceae, Myrsinaceae and Zygophyllaceae, among many others. Quillaja and legumes such as soybeans, beans and peas are a rich source of triterpenoid saponins. The steroidal saponins are typically found in members of the Agavaceae, Alliaceae, Asparagaceae, Dioscoreaceae, Liliaceae, Amaryllidaceae, Bromeliaceae, Palmae and Scrophulariaceae families and accumulate in abundance in crop plants such as yam, alliums, asparagus, fenugreek, Yucca, and Ginseng. The steroidal glycoalkaloids are commonly found in members of the Solanaceae family including tomato, potato, aubergines and Capsicum.

In one embodiment, the subject composition can comprise one or more additional substances and/or nutrients to supplement the needs of the beneficial microorganism of the composition and/or to supplement the needs of soil, water or plants in the environment to which it is applied. These can include, for example, sources of amino acids (including essential amino acids), peptides, proteins, vitamins, microelements, fats, fatty acids, lipids, carbohydrates, sterols, enzymes, and minerals such as calcium, magnesium, phosphorus, potassium, sodium, chlorine, sulfur, chromium, cobalt, copper, iodine, iron, manganese, molybdenum, nickel, selenium, and zinc. In some embodiments, the microorganisms of the composition produce and/or provide these substances.

In certain embodiments, the composition can further comprise one or more carriers and/or excipients suitable for delivery of the composition to soil, water or plants.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents, solubilizers (such as, e.g., Tween 80, Polysorbate 80), colloids, dispersion media, vehicles, fillers (e.g., MCC), chelating agents (such as, e.g., EDTA or glutathione), amino acids (such as, e.g., glycine), proteins, disintegrants, binders, lubricants, wetting agents, emulsifiers, sweeteners, colorants, flavorings, aromatizers, thickeners, coatings, preservatives (such as, e.g., Thimerosal, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like. In certain embodiments, the composition comprises a filler, such as microcrystalline cellulose (MCC).

In certain embodiments, the composition can comprise glucose (e.g., in the form of molasses), glycerol and/or glycerin, as, or in addition to, an osmoticum substance, to promote osmotic pressure during storage and transport of a dry product.

To improve or stabilize the effects of the composition, it can be blended with suitable adjuvants and then used as such or after dilution, if necessary. In preferred embodiments, the composition is formulated as a liquid, a concentrated liquid, or as dry powder or granules that can be mixed with water and other components to form a liquid product.

The compositions can further comprise other compounds and/or methods for efficiently enhancing plant health, growth and/or yields, and/or for supplementing the growth of the beneficial microorganism. For example, in one embodiment, the composition can include and/or can be applied concurrently with nutrients and/or micronutrients for enhancing plant and/or microbe growth, such as magnesium, phosphate, nitrogen, potassium, selenium, calcium, sulfur, iron, copper, and zinc; and/or one or more prebiotics, such as kelp extract, fulvic acid, chitin, humate and/or humic acid. The exact materials and the quantities thereof can be determined by a grower or an agricultural scientist having the benefit of the subject disclosure.

The compositions can also comprise and/or be used in combination with other agricultural compounds and/or crop management systems. In one embodiment, the composition can optionally comprise, or be applied with, for example, natural and/or chemical pesticides, repellants, herbicides, fertilizers, water treatments, non-ionic surfactants and/or soil amendments. If the composition is mixed with compatible chemical additives, the chemicals are preferably diluted with water prior to addition of the subject composition.

The compositions can be formulated into preparations in, for example, solid, semi-solid, liquid or gaseous forms, such as tablets, capsules, pressed pellets, powders, granules, gels, solutions, steaks/spikes (for soil), drops, aerosols, suspensions, concentrates, and other preparations as suitable for a particular application. liquid, dust, granules, microgranules, pellets, wettable powder, flowable powder, emulsions, microcapsules, oils, or aerosols.

### EXEMPLARY EMBODIMENTS

In some embodiments, the composition of the subject invention comprises:
A) one or more microorganisms (yeasts, fungi and/or bacteria) capable of capturing iron and/or producing an iron-capturing growth by-product, wherein preferably at least one of the one or more microorganisms is a Bacillus sp., and wherein even more preferably, the Bacillus sp. is B. subtilis NRRL B-68031 or B. amyloliquefaciens NRRL B-67928;
B) an iron-capturing substance selected from pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexanediamide (DPH), 2,3-dihydroxybenzoic acid, azotochelin ferrichrome, defarasirox, deferiprone, desferrioxamine, fusarinine, chrysobactin, achromobactin, ornibactin, rhodotorulic acid, lysine, glutamic acid, gluconic acid, iron oxyhydroxide minerals, ferrihydrite, magnetite, hematite, geothite, siderite, transferrin, enterobactin, bacillibactin, vibriobactin, azotobactin, aminochelin, pyoverdine, yersiniabactin, agrobactin, staphyloferrin, hydroxamate, catecholate, salicylate, carboxylate, mugineic acid, ferulic acid, caffeic acid, enterobactin, pyoverdine, protochelin, pyochelin, vibrioferrin and/or nicotianamine;
C) a carrier/excipient including solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents, solubilizers (such as, e.g., Tween 80, Polysorbate 80), colloids, dispersion media, vehicles and/or fillers (e.g., MCC);
D) one or more acids selected from, for example, acetic acid, citric acid, formic acid, lactic acid, malic acid, oxalic acid, tartaric acid, uric acid, propionic acid, butyric acid, sorbic acid, fumaric acid, benzoic acid, hydrofluoric acid, caproic acid, salicylic acid, gluconic acid, pyruvic acid, adipic acid, trichloroacetic acid, glycolic acid, cinnamic acid, carboxylic acids, succinic acid, carbonic acid, glutaric acid, decanoic acid, ascorbic acid, hydrochloric acid, sulfuric acid, nitric acid, phosphoric acid, boric acid, perchloric acid, hydrofluoric acid, hydrobromic acid, and sulfonic acid, in an amount suitable for adjusting the pH of the composition or the environment to which it is applied to 6.8 or lower, preferably, 5.0 or lower, more preferably, 4.8 or lower; and/or
E) a biosurfactant selected from sophorolipids, rhamnolipids, cellobiose lipids, mannosylerythritol lipids, trehalose lipids, surfactin, iturin, arthrofactin, viscosin, fengycin, and lichenysin.

In some embodiments, the composition comprises each of components A-E. In some embodiments, the composition comprises any combination of A-E, or any one of A-E individually.

In some embodiments, the composition comprises spore, vegetative and/or biofilm-form B. subtilis NRRL B-68031 or B. amyloliquefaciens NRRI, B-67928; and pulcherrimin and/or pulcherriminic acid.

In some embodiments, the composition comprises spore, vegetative and/or biofilm-form B. subtilis NRRI. B-68031 or B. amyloliquefaciens NRRL B-67928; and a carrier/excipient.

In some embodiments, the composition comprises spore, vegetative and/or biofilm-form B. subtilis NRRL B-68031 or B. amyloliquefaciens NRRL B-67928; and one or more acids listed in point D).

In some embodiments, the composition comprises spore, vegetative and/or biofilm-form B. subtilis NRRI. B-68031 or B. amyloliquefaciens NRRL B-67928; pulcherrimin and/or pulcherriminic acid; and a carrier/excipient.

In some embodiments, the composition comprises spore, vegetative and/or biofilm-form B. subtilis NRRL B-68031 or B. amyloliquefaciens NRRL B-67928; pulcherrimin and/or pulcherriminic acid; a carrier; and one or more acids listed in point D).

In some embodiments, the composition comprises spore, vegetative and/or biofilm-form B. subtilis NRRI B-68031 or B. amyloliquefaciens NRRI, B-67928; pulcherrimin and/or pulcherriminic acid; a carrier; and a biosurfactant.

Production of Microorganisms and/or Microbial Growth By-Products

The subject invention utilizes methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

As used herein “fermentation” refers to cultivation or growth of cells under controlled conditions. The growth could be aerobic or anaerobic. In preferred embodiments, the microorganisms are grown using SSF and/or modified versions thereof.

In one embodiment, the subject invention provides materials and methods for the production of biomass (e.g., viable cellular material), extracellular metabolites, residual nutrients and/or intracellular components.

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In one embodiment, one or more biostimulants may also be included, meaning substances that enhance the rate of growth of a microorganism. Biostimulants may be species-specific or may enhance the rate of growth of a variety of species.

In some embodiments, the method for cultivation may further comprise adding an antimicrobial in the medium before, and/or during the cultivation process.

In certain embodiments, an antibiotic can be added to a culture at low concentrations to produce microbes that are resistant to the antibiotic. The microbes that survive exposure to the antibiotic are selected and iteratively re-cultivated in the presence of progressively higher concentrations of the antibiotic to obtain a culture that is resistant to the antibiotic. This can be performed in a laboratory setting or industrial scale using methods known in the microbiological arts. In certain embodiments, the amount of antibiotic in the culture begins at, for example, 0.0001 ppm and increases by about 0.001 to 0.1 ppm each iteration until the concentration in the culture is equal to, or about equal to, the dosage that would typically be applied to a iron-capturing pathogen.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

The biomass content of the fermentation medium may be, for example, from 5 g/l to 180 g/l or more, or from 10 g/l to 150 g/l. The cell concentration may be, for example, at least 1×10⁹, 1×10¹⁰, 1×10¹¹, 1×10¹² or 1×10¹³ cells per gram of final product.

The microbial growth by-product produced by microorganisms of interest may be retained in the microorganisms or secreted into the growth medium. The medium may contain compounds that stabilize the activity of microbial growth by-product.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

### Preparation of Microbe-Based Products

A “microbe-based product,” is a product to be applied in practice to achieve a desired result. The microbe-based product can be simply a microbe-based composition harvested from a microbe cultivation process. Alternatively, a microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

One microbe-based product of the subject invention is simply the fermentation medium containing a microorganism and/or the microbial metabolites produced by the microorganism and/or any residual nutrients. The product of fermentation may be used directly without extraction or purification. If desired, extraction and purification can be easily achieved using standard extraction and/or purification methods or techniques described in the literature.

The microorganisms in the microbe-based product may be in an active or inactive form. Furthermore, the microorganisms may be removed from the composition, and the residual culture utilized. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or medium (e.g., broth or solid substrate) resulting from the microbial growth can be removed from the growth vessel and transferred via, for example, piping for immediate use.

In one embodiment, the microbe-based product is simply the growth by-products of the microorganism. For example, biosurfactants produced by a microorganism can be collected from a submerged fermentation vessel in crude form, comprising, for example about 50% pure biosurfactant in liquid broth.

In other embodiments, the microbe-based product (microbes, medium, or microbes and medium) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

Upon harvesting, for example, the yeast fermentation product, from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, solvents, biocides, other microbes and other ingredients specific for an intended use.

Other suitable additives, which may be contained in the formulations according to the invention, include substances that are customarily used for such preparations. Examples of such additives include surfactants, emulsifying agents, lubricants, buffering agents, solubility controlling agents, pH adjusting agents, preservatives, stabilizers and ultra-violet light resistant agents.

In one embodiment, the product may further comprise buffering agents including organic and amino acids or their salts. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

In one embodiment, additional components such as an aqueous preparation of a salt, such as sodium bicarbonate or carbonate, sodium sulfate, sodium phosphate, or sodium biphosphate, can be included in the formulation.

Advantageously, in accordance with the subject invention, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

Methods for Treating and/or Preventing Iron Deficiency in Plants

The subject invention provides methods for treating iron deficiencies and iron chlorosis in plants, wherein a composition according to the subject invention is applied to a plant and/or its surrounding environment. In certain embodiments, the method works by enhancing the bioavailability of iron and other nutrients to a plant. Accordingly, the methods can also be used for increasing nutrient uptake by plants.

As used herein, “applying” a composition or product refers to contacting a composition or product with a target or site such that the composition or product can have an effect on that target or site. The effect can be due to, for example, microbial growth and/or interaction with a plant, as well as the action of a metabolite, enzyme, biosurfactant or other microbial growth by-product. Applying can also include “treating” a target or site with a composition. In some embodiments, multiple plants and/or their surrounding environments are treated according to the subject methods.

A plant's “surrounding environment” means the soil and/or other medium in which the plant is growing, which can include the rhizosphere. In certain embodiments, the surrounding environment does not extend past, for example, a radius of at least 5 miles, 1 mile, 1,000 feet, 500 feet, 300 feet, 100 feet, 10 feet, 8 feet, or 6 feet from the plant.

In some embodiments, the method comprises applying a composition according to the subject invention alongside an acid, wherein the acid modulates the pH of the composition or the environment to 6.8 or lower, preferably 5.0 or lower, more preferably 4.8 or lower. The acid can be an organic acid selected from, for example, acetic acid, citric acid, formic acid, lactic acid, malic acid, oxalic acid, tartaric acid, uric acid, propionic acid, butyric acid, sorbic acid, fumaric acid, benzoic acid, hydrofluoric acid, caproic acid, salicylic acid, gluconic acid, pyruvic acid, adipic acid, trichloroacetic acid, glycolic acid, cinnamic acid, carboxylic acids, succinic acid, carbonic acid, glutaric acid, decanoic acid, and ascorbic acid. The acid can also be an inorganic acid selected from, for example, hydrochloric acid, sulfuric acid, nitric acid, phosphoric acid, boric acid, perchloric acid, hydrofluoric acid, hydrobromic acid, and sulfonic acid.

In some embodiments, the method comprises applying a composition according to the subject invention alongside an iron chelate and/or iron sulfate.

If present in the composition, microbes can be either live (or viable) or inactive at the time of application. In some embodiments, the microbes are in the form of yeast extract and/or another microbial hydrolysate.

Microbial growth by-products can be applied in addition to the growth by-products produced by the microorganism(s) of the composition, or they can be applied on their own without the microorganism(s).

The methods can further comprise adding materials to enhance microbe growth during application (e.g., adding nutrients and/or prebiotics). Thus, live microorganisms can grow in situ and produce the active compounds onsite. Consequently, a high concentration of microorganisms and their growth by-products can be achieved easily and continuously in an environment.

In some embodiments, the method comprises applying an entire microbial culture, comprising inactivated cells in submerged or solid-state fermentation medium. Advantageously, this reduces the amount of waste products produced during production of the subject compositions while increasing efficiency of production by removing the steps of extraction and/or purification of microbial metabolites. Furthermore, inclusion of inactive cells and residual fermentation medium provides rich sources of organic and inorganic nutrients that are essential for supporting soil and/or plant health.

In one embodiment the composition is dispersed in an environment while being supported on a carrier. The carrier can be made of materials that can retain microorganisms thereon relatively mildly and thus allow easy release of microorganisms thus proliferated. The carrier is preferably inexpensive and in some embodiments can act as a nutrient source for the microorganisms thus applied, particularly a nutrient source that can be gradually released. Biodegradable carrier materials include cornhusk, sugar industry waste, or any agricultural waste. The water content of the carrier typically varies from 1% to 99% by weight, preferably from 5% to 90% by weight, more preferably from 10% to 85% by weight.

Application can include contacting the microbe-based product directly with a plant, plant part, and/or the plant's surrounding environment (e.g., the soil or the rhizosphere). The microbe-product can be applied as a seed treatment or to the soil surface, or to the surface of a plant or plant part (e.g., to the surface of the roots, tubers, stems, flowers, leaves, fruit, or flowers). It can be sprayed, poured, sprinkled, injected or spread as liquid, dry powder, dust, granules, microgranules, pellets, wettable powder, flowable powder, emulsions, microcapsules, oils, gels, pastes or aerosols.

In some embodiments, the composition is contacted with one or more roots of the plant. The composition can be applied directly to the roots, e.g., by spraying or dunking the roots, and/or indirectly, e.g., by administering the composition to the soil in which the plant grows (e.g., the rhizosphere). The composition can be applied to the seeds of the plant prior to or at the time of planting, or to any other part of the plant and/or its surrounding environment.

In one embodiment, the composition may be worked into the soil using a mechanical action, for example, by tilling. In still further embodiments, the application of a composition may be subsequently followed by application of a liquid, such as water. The water may be applied as a spray, using standard methods known to one of ordinary skill in the art. Other liquid wetting agents and wetting formulations may also be used.

In some embodiments, the composition is applied to the soil surface without mechanical incorporation. The beneficial effect of the soil application can be activated by rainfall, sprinkler, flood, or drip irrigation, and subsequently delivered to, for example, the roots of plants.

In some embodiments, the compositions, either in a dry or in liquid formulation, are applied as a seed treatment or to the surface of a plant or plant part (e.g., to the surface of a plant's leaves or roots).

In some embodiments, the composition is contacted with the leaves of the plant, e.g., by spraying. In some embodiments, the composition is injected or otherwise implanted into the stem or trunk of the plant such that the composition is made available to the internal and vascular tissue of the plant.

Plants and/or their environments can be treated at any point during the process of cultivating the plant. For example, the soil treatment composition can be applied to the soil prior to, concurrently with, or after the time when seeds are planted therein. It can also be applied at any point thereafter during the development and growth of the plant, including when the plant is flowering, fruiting, and during and/or after abscission of leaves.

In one embodiment, the method can be used in a large scale agricultural setting. The method can comprise administering the composition into a tank connected to an irrigation system used for supplying water, fertilizers or other liquid compositions to a crop, orchard or field. Thus, the plant and/or soil surrounding the plant can be treated with the composition via, for example, soil injection, soil drenching, or using a center pivot irrigation system, or with a spray over the seed furrow, or with sprinklers or drip irrigators. Advantageously, the method is suitable for treating hundreds of acres of crops, orchards or fields at one time.

In one embodiment, the method can be used in a smaller scale setting, such as in a home garden or greenhouse. In such cases, the method can comprise spraying a plant and/or its surrounding environment with the composition using a handheld lawn and garden sprayer. The composition can be mixed with water, and optionally, other lawn and garden treatments, such as fertilizers and pesticides. The composition can also be mixed in a standard handheld watering can and poured onto soil.

The subject compositions and methods can be used either alone or in combination with other methods and compounds for efficient enhancement of plant health, growth and/or yields, as well as other compounds for efficient treatment and prevention of plant pathogenic pests. For example, the methods can be used concurrently with sources of nutrients and/or micronutrients for enhancing plant and/or microbe growth, such as magnesium, phosphate, nitrogen, potassium, selenium, calcium, sulfur, iron, copper, and zinc; and/or one or more prebiotics, such as kelp extract, fulvic acid, chitin, humate and/or humic acid. The exact materials and the quantities thereof can be determined by a grower or an agricultural scientist having the benefit of the subject disclosure.

The compositions can also be used in combination with other agricultural compounds and/or crop management systems. In one embodiment, the composition can optionally comprise, and/or be applied with, for example, natural and/or chemical pesticides, repellants, herbicides, fertilizers, water treatments, non-ionic surfactants and/or soil amendments.

In one embodiment, the method can be used to inoculate a rhizosphere with one or more beneficial microorganisms. For example, in preferred embodiments, the microbes of the composition can colonize the rhizosphere and, in addition to enhancing the amount of bioavailable iron for a plant, provide multiple benefits to the plant whose roots are growing therein, including protection and nourishment.

Advantageously, the subject methods can be used to enhance health, growth, and/or yields in plants having compromised immune health due to an infection from a pathogenic agent or from an environmental stressor, such as, for example, drought. Thus, in certain embodiments, the subject methods can also be used for improving the immune health, or immune response, of plants.

The methods can also help enhance agricultural yields, even in depleted or damaged soils; restore depleted greenspaces, such as pastures, forests, wetlands and prairies; and restore uncultivatable land so that it can be used for farming, reforestation and/or natural regrowth of plant ecosystems. Additionally, through improved agricultural practices, the methods can help reduce pollution caused by emissions of greenhouse gases.

In certain embodiments, enhancing soil health comprises improving one or more qualities of soil. This can comprise, for example, removing and/or reducing pollutants in the soil, improving the nutrient content and nutrient availability of the soil, improving drainage and/or moisture retention properties of the soil, improving the salinity of the soil, improving the soil microbiome diversity, and/or controlling a soil-borne pest. Other improvements can include adding bulk and/or structure to soils that have been eroded by wind and/or water, as well as preventing and/or delaying erosion of soil by wind and/or water.

In certain embodiments, the methods comprise a step of characterizing the soil type and/or soil health status prior to treating the soil according to the subject methods. Accordingly, the method can also comprise tailoring the composition in order to meet a specific soil type and/or soil health need. Methods of characterizing soils are known in the agronomic arts.

In some embodiments, the methods are used for restoring soil health, wherein the soil being treated was once healthy, but deteriorated over some period of time. The restoration may bring the soil back to its previous state of health and/or an enhanced state of health.

In certain embodiments, the method results in removal and/or reduction of pollutants from soil, including remediation of soils contaminated with iron. In some embodiments, the pollutants are degraded directly by the applied microorganisms of the composition.

In certain embodiments, the methods can also help improve soil microbiome diversity by promoting colonization of the soil and plant roots growing therein with beneficial soil microorganisms. Growth of nutrient-fixing microbes, such as rhizobium and/or mycorrhizae, can be promoted, as well as other endogenous and applied microbes, thereby increasing the number of different species within the soil microbiome.

In one embodiment, the subject invention can also be used for improving one or more qualities of soil, thereby enhancing the performance of the soils for agricultural, home and gardening purposes.

In certain embodiments, the soil treatment composition may also be applied so as to promote colonization of the roots and/or rhizosphere as well as the vascular system of the plant in order to enhance plant health and vitality. Thus, nutrient-fixing microbes can be promoted, as well as other beneficial endogenous and exogenous microbes, and/or their by-products that promote crop growth, health and/or yield.

In one embodiment, the method can be used for enhancing penetration of beneficial molecules through the outer layers of root cells.

The subject invention can be used to improve any number of qualities in any type of soil, for example, clay, sandy, silty, peaty, chalky, loam soil, and/or combinations thereof. Furthermore, the methods and compositions can be used for improving the quality of dry, waterlogged, porous, depleted, compacted soils and/or combinations thereof.

In one embodiment, the method can be used for improving the drainage and/or dispersal of water in waterlogged soils. In one embodiment, the method can be used for improving water retention in dry soil.

In one embodiment, the method can be used for improving nutrient retention in porous and/or depleted soils.

All plants and plant parts can be treated in accordance with the invention. In this context, plants are understood as meaning all plants and plant populations such as desired and undesired wild plants or crop plants (including naturally occurring crop plants). Crop plants can be plants that can be obtained by traditional breeding and optimization methods or by biotechnological and recombinant methods, or combinations of these methods, including the transgenic plants and the plant varieties.

Plant parts are understood as meaning all aerial and subterranean parts and organs of the plants such as shoot, leaf, flower and root, examples which may be mentioned being leaves, needles, stalks, stems, flowers, fruit bodies, fruits and seeds, but also roots, tubers and rhizomes. The plant parts also include crop material and vegetative and generative propagation material, for example cuttings, tubers, rhizomes, slips and seeds.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—pH Growth Testing of B4 Strain

The B4 strain was spread on Tryptic Soy Agar (TSA) at a neutral (6.8) and acidic (4.8) pH to look for differences in growth, with the goal of determining how it would behave in the various pH environments within a cow's digestive system.

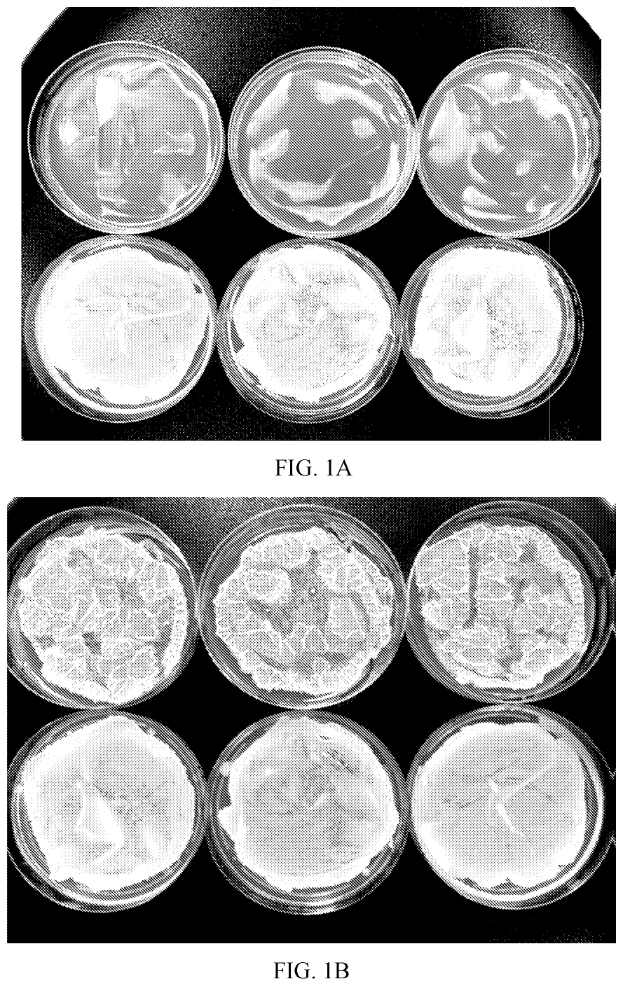

Growth at neutral pH (6.8) was faster within 24 hours compared to the acidic plates, but at 48 hours, the growth on pH 4.8 agar was equal or greater. FIGS. 1A-1B. Additionally, a significant amount of exopolysaccharide (EPS) was produced when grown on an acidic agar. FIG. 1C. This is a result of environmental stress.

The dried B4 spores were also added in sterile PBS adjusted to pH 2.8 and left for 24 hours. The same was then plated on neutral (6.8) TSA plates. A lawn of growth was present, but no EPS was produced (similar to pH 6.8 plates from FIG. 1). This shows that dried spores exposed to an overall harsh environment were still intact and viable. Overall, the pH of the growing media/environment influences EPS production.

### Example 2—EPS

B4 was grown in a liquid medium at pH 4.8 specifically for the production of EPS. The assumed EPS was isolated out of the culture and purified.

FTIR analysis confirmed the purified sample to be an EPS and HPLC analysis confirmed the existence of a large peak denoting a sugar oligomer. A C═O bond was also observed via UV absorption.

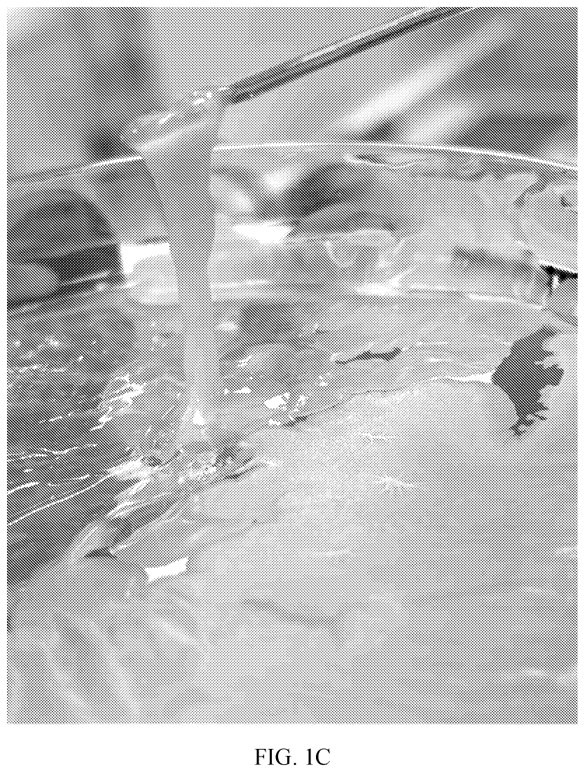

Additionally, and surprisingly, when the culture was processed for EPS extraction, there was a purple stripe present in the cell pellet. When the EPS purification was completed, the sample was a pink color, suggesting the presence of pulcherrimin or pulcherriminic acid. FIG. 2.

### Example 3—Enzyme Assay Testing-Amylase

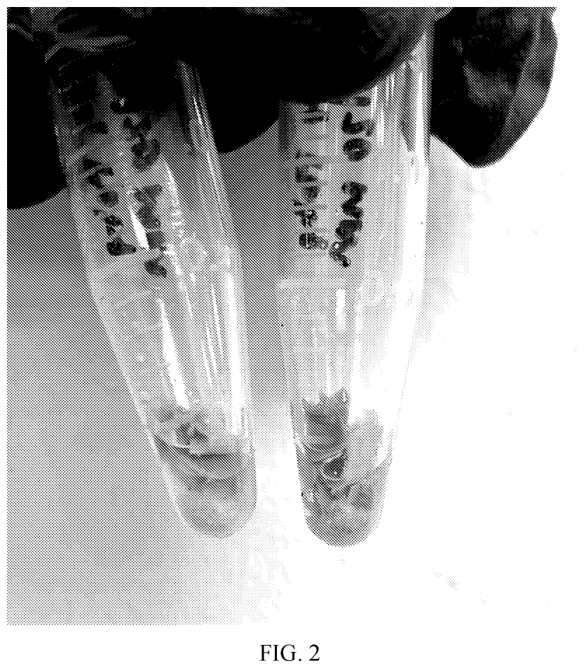

Amylase is an enzyme that hydrolyzes the glycosidic bonds in starch molecules by converting complex carbohydrates to simple sugars. Agar, a starch, was inoculated with B4 and incubated for growth. FIG. 3. After running the plate assay, an orange color around the bacterial growth was observed, indicating the breakdown of starch. This is a positive amylase test.

### Example 4—Enzyme Assay Testing-Cellulase

Cellulases are enzymes that convert cellulose to glucose. B4 was tested for cellulase activity using carboxymethylcellulose agar (CMCA) media plates.

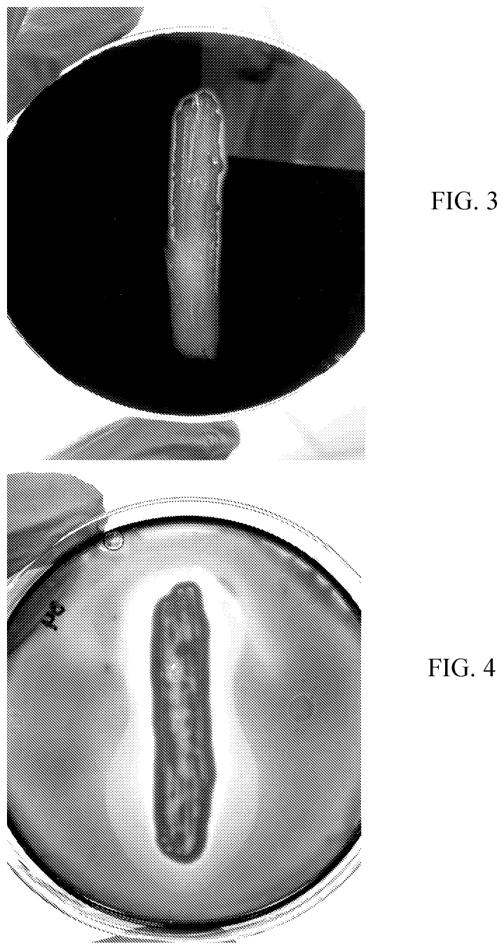

B4 was grown in tryptic soy broth with and without cellobiose added. After 48 hours of growth, the liquid culture was streaked onto CMCA plates. After inoculation, and once growth was present on the agar, an iodine solution was introduced to the plates. A yellow zone of clearing around the bacterial growth indicates the breakdown of cellulose and the presence of cellulase enzymes. FIG. 4.

### Example 5—Detection of Siderophores

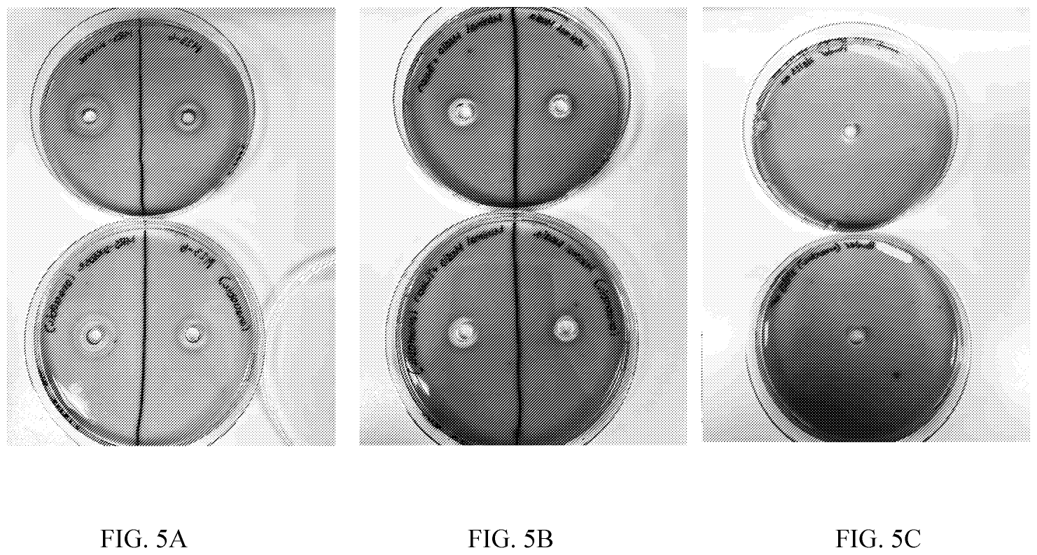

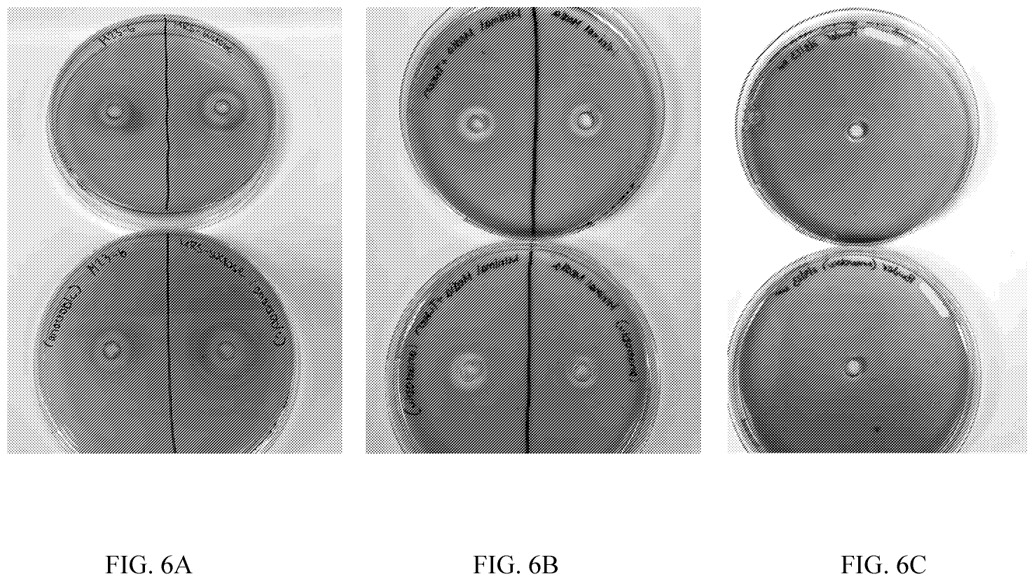

Chrome azurol S (CAS) assay was used for the detection of siderophores from B4 cultures and dried B4 spores. The tests were run in aerobic and anaerobic environments on different growth media: MRS-sucrose, M23-6, minimal media with Tween, and minimal media without Tween. The plates were observed at 6 hours (FIGS. 5A-5C) and 24 hours (FIGS. 6A-6C).

Siderophores scavenge iron from an Fe-CAS-hexadecyltrimethylammonium bromide complex, and the release of the CAS dye results in a color change from blue to orange. Any observable color change on CAS agar plates indicates a qualitative detection of siderophores.

All B4 media cultures tested were positive for siderophore production and activity. MRS-sucrose (the richest media) produced the strongest activity. There were no differences in siderophore activity between aerobic and anaerobic conditions with the four media cultures.

Dried B4 spores produced less siderophore activity compared to the B4 cultures, which may due to spore dormancy. The dried spores performed better in aerobic conditions.

### Example 6—Iron Activity Assay

To understand how B4 interacts with iron, and to determine how iron activity differs between culture media, an iron assay kit (Sigma-Aldrich) was used to determine the concentration of ferrous (Fe²⁺), ferric (Fe³⁺) and total iron present in different B4 cultures.

Iron is released from the sample by the addition of an acidic buffer. Released iron is reacted with chromagen, resulting in a colorimetric (593 nm) product that is proportional to the iron present.

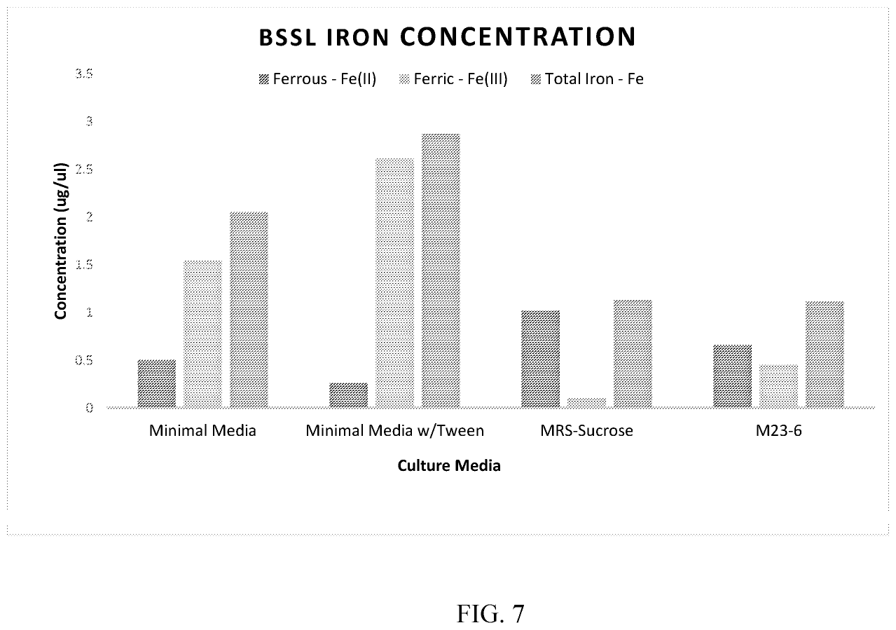

The results in FIG. 7 correlate with the siderophore results reported in FIGS. 5-6, meaning higher siderophore activity correlates with lower total iron levels. For example, MRS-sucrose media is the richest media with the highest siderophore activity and the lowest total iron levels. Additionally, the minimal media used is designed for increased production of pulcherrimin production, which is a ferric chelate. Greater levels of pulcherrimin in the culture should increase ferric and total iron concentrations.

1. A method for treating and/or preventing iron deficiency and/or iron chlorosis in a plant, wherein the method comprises applying, to the plant and/or its surrounding environment, an iron-capturing agent, wherein the iron-capturing agent is an iron-capturing non-pathogenic Bacillus sp. bacterium and/or an iron-capturing growth by-product thereof.

2. The method of claim 1, further comprising applying an acid to the environment to achieve a pH of 6.8 or less.

3. The method of claim 1, wherein the composition is applied via an irrigation system or via a sprayer applicator to soil in which the plant is growing.

4. (canceled)

5. The method of claim 1, wherein the composition is applied directly to the plant.

6. The method of claim 5, wherein the composition is injected or implanted into the plant's stem or trunk.

7. The method of claim 5, wherein the composition is applied as a seed treatment.

8-10. (canceled)

11. The method of claim 1, wherein the microorganism is Bacillus subtilis B4 NRRL B-68031 or Bacillus amyloliquefaciens NRRL B-67928.

12. The method of claim 91, wherein the microorganism is administered in spore form, but grows in biofilm form upon contact with an acidic environment having pH 6.8 or less.

13. The method of claim 1, comprising applying a further iron-capturing agent selected from pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexancdiamide (DPH), 2,3-dihydroxybenzoic acid, azotochelin, transferrin, enterobactin, pyoverdine, protochelin, pyochelin, bacillibactin, vibriobactin, vibrioferrin azotobactin, aminochelin, yersiniabactin, agrobactin, staphyloferrin, ferrichrome, defarasirox, deferiprone, desferrioxaminc, fusarinine, chrysobactin, achromobactin, ornibactin, rhodotorulic acid, lysine, glutamic acid, gluconic acid, iron oxyhydroxide minerals, ferrihydrite, magnetite, hematite, geothite, sideritehydroxamate, catecholates, salicylates, carboxylates, mugineic acid, ferulic acid, caffeic acid, and/or nicotianamine.

14-15. (canceled)

16. A composition for treating and/or preventing iron deficiency and/or iron chlorosis, the composition comprising an iron-capturing agent, wherein the iron-capturing agent is a non-pathogenic Bacillus sp. bacterium and/or an iron capturing growth by-product thereof.

17. The composition of claim 16, having a pH of 6.8 or less.

18-19. (canceled)

20. The composition of claim 16, wherein the microorganism is Bacillus subtilis B4 NRRL B-68031 or Bacillus amyloliquefaciens NRRL B-67928.

21. The composition of claim 16, wherein the microorganism is in spore form or biofilm form.

22. The composition of claim 16, wherein the composition comprises a further iron-capturing agent selected from pulcherrimin, pulcherriminic acid, citrate, citric acid, EDTA (Ethylenediaminetetraacetic acid), ferric EDTA, DTPA (Diethylenetriaminepentaacetic acid), EDDHA (Ethylenediamine di(o-hydroxyphenylacetic acid), N,N-dihydroxy-N,N′-diisopropylhexanediamide (DPH), 2,3-dihydroxybenzoic acid, azotochelin, transferrin, enterobactin, pyoverdine, protochelin, pyochelin, bacillibactin, vibriobactin, vibrioferrin azotobactin, aminochelin, yersiniabactin, ferrichrome, defarasirox, deferiprone, desferrioxamine, fusarinine, chrysobactin, achromobactin, ornibactin, rhodotorulic acid, lysine, glutamic acid, gluconic acid, iron oxyhydroxide minerals, ferrihydrite, magnetite, hematite, geothite, sideritehydroxamate, catecholates, salicylates, carboxylates, mugineic acid, ferulic acid, caffeic acid, and/or nicotianamine.

23. The composition of claim 16, further comprising a germination enhancer, wherein the germination enhancer is L-alanine, L-leucine, or manganese.

24. The composition of claim 16, wherein the composition comprises a microbial growth by-product selected from biosurfactants, enzymes, organic acids, fatty acids, amino acids, proteins, peptides, alcohols, polyketides, natural antibiotics, aldehydes, amines, sterols, and vitamins.

25. The composition of claim 16, further comprising a nutrient or prebiotic.
